(19) United States
(12) Patent Application Publication
(10) Pub. No.: US 2014/0220071 A1
(43) Pub. Date: Aug. 7, 2014

(54) BIOLOGICALLY ACTIVE FOOD ADDITIVE FOR NORMALIZING THE FUNCTION OF THE THYROID GLAND

(71) Applicants: Vyacheslav N. Trifonov, Zarechny (RU); Yulia A. Elistratova, Penza (RU); Konstantin G. Elistratov, Penza (RU); Natalia V. Kurus', Penza (RU)

(72) Inventors: Vyacheslav N. Trifonov, Zarechny (RU); Yulia A. Elistratova, Penza (RU); Konstantin G. Elistratov, Penza (RU); Natalia V. Kurus', Penza (RU)

(73) Assignee: JSC Patent Attorney

(21) Appl. No.: 14/238,322

(22) PCT Filed: Apr. 10, 2012
(86) PCT No.: PCT/RU2012/000269

§ 371 Date: Apr. 3, 2014

(30) Foreign Application Priority Data
Mar. 3, 2011 (RU) 2011108624

(51) Int. Cl. A61K 36/73; A61K 36/03; A61K 36/28

(52) U.S. Cl. CPC A61K 36/73; A61K 36/28; A61K 36/03
USPC 424/195.17

(57) ABSTRACT

An active biologically active dietary additive for the normalization of the thyroid gland function contains roots, rhizomes or the herb of white cinquefoil, or their mixture, Echinacea purpurea, and Laminaria, the ratio in mass percent being: white cinquefoil—10-75, Echinacea purpurea—10-50, Laminaria—10-80.

### CROSS-REFERENCE TO RELATED APPLICATIONS

This application is a U.S. National phase application of the International application PCT/RU2012/000269, filed Apr. 10, 2012, the entire content of the International application being hereby incorporated in its entirety into the present application by reference

### BACKGROUND OF THE DISCLOSURE

1. Field of Technology

This disclosure relates to a food processing industry, precisely to biologically active dietary additives (BAA) for the normalization of the thyroid function.

2. Description of Related Art

A prior art search on this matter uncovered patent RU2333764 for “Medication for the treatment and prevention of the disease of thyroid gland”, containing roots and rhizomes of white cinquefoil (Potentilla alba L.), herb of bur beggar-ticks (Bidens tripartite L.) and roots of common licorice (Glycyrrhiza glabra L.).

Also known is Russian patent RU2351354 for “Medication for the treatment and prevention of the disease of thyroid gland”. The medication is a tea mix comprising white cinquefoil, roots of common licorice, herb of bur beggar-ticks and thallomes of Laminaria saccharina, with the following component ratio—20:5:8:7, respectively, the white cinquefoil being present in the form of roots, rhizomes or herb or a mixture thereof, the forms of the medication including ground herb powder, alcohol-water tincture, liquid and dry extractions.

### BRIEF SUMMARY

The object of the disclosure is widening the range of biologically active dietary additives, possessing a wide spectrum of effect on the human body and contributing to the health improvement of the population.

This problem is solved by providing a biologically active dietary additive for the normalization of the thyroid function comprising, according to the disclosure, contains roots and rhizomes of white cinquefoil or herb of white cinquefoil, or their mixture, as well as purple coneflower and Laminaria, with the following ratio of the components in mass percent %: roots and rhizomes of white cinquefoil, or the herb of white cinquefoil,

or their mixture 10-75,
purple coneflower 10-50,

### Laminaria 10-80.

The technical result of the additive is providing a biologically active composition with higher bioavailability, that has optimally selected component ratio and shows no side effects, to thus allow using it in the most effective way for the prevention of diseases of the thyroid gland and the raise of body immunological status.

### DETAILED DESCRIPTION

Thyroid disorders are accompanied by an immunity decrease; therefore, it was suggested to intensify the therapeutic effect of the white cinquefoil by the action of Echinacea purpurea. For that reason, “Parapharm” Ltd. offers to release biologically active additive “TIREO-VIT”.

“Tireo-Vit” comprises white cinquefoil, that shows its antibacterial activity, contains a great number of macro- and microelements, normalizes the thyroid function, and it is used for the prevention of such thyroid disorders as thyrotoxicosis, hyperthyroidism, hyperplasia and hypothyroidism. There are two effective agents in the composition: elemental iodine and anion of iodous acid that is contained in the root of a white cinquefoil, as well as polysaccharides of Echinacea purpurea that have an immunopotentiating activity.

It is recommended to use as a biologically active dietary additive in the case of different thyroid disorders such as: hypothyroidism (hypofunction), hyperthyroidism (hyperfunction, thyrotoxicosis, Basedow-Graves, DTG (diffuse toxic goiter) disease), autoimmune thyroditis (Hashimoto thyroiditis, CAT), euthyroid goiter (diffusive, nodular/multinodular), hyperplasia of thyroid gland.

Additional properties of medicinal plants that the “TIREO-VIT” comprises are as follows:

White cinquefoil contains carbohydrates (amylum), iridoids, saponins, phenolcarbonic acids, flavonoids (quercetine) and tanning agents. The unique value of the plant is that in terms of micro- and macroelements it contains almost all Mendeleev's Periodic Table. First of all, a mineral balance and salt exchange within the body turn to normal while using a White cinquefoil, a hormonal balance straightens; metabolic processes normalize. All agents contained in cinquefoil are not toxic and do not have any side effects on the body.

Specifically, flavonoids (vitamin P remedies) have a positive impact on the blood vessel walls, regulating their ductance, eliminating their brittleness and fragility; they reduce heart rate and increase their amplitude; reduce arterial tension, have effect on the blood count, stimulating the production of erythrocytes and leukocytes, and reducing the level of cholesterol; they intensify a bile flow and increase the tone of intestinal canal.

Further, phenolcarbonic acids (pyrocatechins, hydroquinones and pyrogallols) have a broad capacity for a reversible oxidation with the formation of semiquinone radicals and quinones, thereby they easily react with thiol groups of proteins; particularly, their influence on vascular and cellular permeability might be explained by the cooperation of these agents with proteins of cellular membranes and vassal-tissue barriers.

Yet further, iridoids-phytochemicals provide a therapeutic effect in case of thyroid gland disorders. It has been proved, that iridoids kill very harmful free radicals, maintain normal level of cholesterol, increase energy, stimulate cardiac performance, reinforce immune system, guard the body against various kinds of inflammations, keep the cells away from mutation, and increase healthy activity of the brain.

White cinquefoil is a reservoir of Mn, Zn, Cu, Se, Co, Fe, Si, Al. White cinquefoil demonstrates antibacterial activity, and for that reason it is applied in case of colitis, enterocolitis, dysentery, diarrhea, gastrointestinal torminas as anastaltic and hemostatic remedy. Besides that, herbalists recommend to use white cinquefoil for preventive treatment and treatment of liver diseases, disorders in cardiovascular system and gastrointestinal tract, particularly, ulcer, as well as an antiseptic and wound-healing remedy. White cinquefoil is used in the case of arthragra and rheumatism, it is a good blood depurant. White cinquefoil is a reliable remedy in the case of ovarian dysfunction, descent of womb and dysmenorrhea. The intake of white cinquefoil regulates arterial blood pressure; normalizes a body weight; regulates calcium metabolism to thereby improve the condition of skin, hair and nails. It contributes to the resolution of soft tumors, nodular lumps; it has been proved that white cinquefoil contributes to the excretion of radiation, lowers the level of cholesterol, resolves cysts and myomas.

Echinacea purpurea contains essential oils, glycosides, amides, antibiotic polyacetylenes, inulin, B-group vitamins, iron that is needed to form red corpuscules, calcium needed for strong bones and teeth, selenium for the capacity to resist diseases, as well as silicium that is needed to form tissues. Echinacea is used in the case of disorders associated with a reduction of the functional status of immune system and induced by chronic inflammatory diseases, exposure to ionizing radiation, ultraviolet rays, chemotherapeutic compounds and long-term antibiotic therapy. In some metabolic disorders (diabetes and hepatic disorders), influence of various chemical compounds of toxic nature (such as heavy metals, pesticides, insecticides and fungicides), contained in the air and food substances, the intake of Echinacea containing preparations stimulates the immune system. Aditionally, Echinacea preparations have antibacterial, anti-viral and anti-mycotic properties. Echinacea preparations inhibit the growth and reproduction of streptococcus, staphylococcus, Escherichia coli, viruses of influenza, herpes, stomatitis; they are effective in the case of inflammatory diseases (rheumatism, polyarthritis, prostatitis and gynecological disorders), upper air passages diseases; in various wound processes (trophic ulcers and central osteitis) and microbial eczema.

Laminaria (sea girdle)—iodine (around 3%) in a form of iodine-organic compounds and iodides; vitamins A, B₁, B₂, B₁₂, C, and O, folic acid, carbohydrates (polysaccharide aminarine, mannitol, alginic acid), aging pigment, bromine salts, traces of arsenic, salts of potassium, sodium, calcium, magnesium, copper, and cobalt.

Due to the great content of iodine and vitamins it is used in a form of powder for the treatment and prophylaxis of atherosclerosis and goiter.

In oncological clinic it has been uncovered that the powder of laminaria contributes to the improvement of a general state of the patients and frame of their mind (A. D. Turova, A. S. Gladkikh, 1965).

It is also recommended in case of chronic constipations as eccoprotic (2-3 g of powder for 150 ml of water, nocte), as well as for the improvement of metabolic process and in the case of arthragra.

In China and Japan, laminaria is used in treatment and prophylaxis of the thyroid disorders.

Combined administration of white cinquefoil and Echinacea is highly reasonable. Common usage of the specified plants in thyroid function disorders is not known in the art.

Both plants increase energy and they are immune response modifiers, activators of cell-mediated immunity and intrinsic cellular metabolism. Common use of these two plants will substantially intensify recited pharmacological effects. Particularly, a maximum activity of Echinacea appears strictly subjected to availability of bioflavonoids which are contained in white cinquefoil.

Incorporation of Laminaria into the preparation composition results from the fact that Laminaria is rich with organic iodine which is needed to achieve a maximum of therapeutic effect, especially in case of hypothyroidism and euthyroid goiter.

It has been found in the course of numerous experiments that to receive an additive with the supreme bioavailability of all included components, as well as for the purpose of obtaining the maximum efficacy of this additive in respect of prevention of thyroid gland disorders and raising the immunological status, the ratio of the compounds has to be the following: white cinquefoil (roots and rhizomes or the herb, or their mixture)—10-75 in mass percent (%), Echinacea purpurea 10-50 in mass percent (%), Laminaria 10-80 in mass percent (%).

The preparation has been illustrated with the following examples:

### EXAMPLE 1

To prepare BAA the compounds are combined in the ratio: 50 mg of milled roots of white cinquefoil, 10 mg milled herb of Echinacea purpurea and 10 mg of milled thallomes of Laminaria. All ingredients are thoroughly mixed. The resultant mixture is pelletized.

### EXAMPLE 2

To prepare BAA, the components are combined in the following ratio: Leaves of white cinquefoil—50 mg, herb of Echinacea purpurea—50 mg and thallomes of Laminaria—100 mg. All ingredients are milled and extensively mixed. The resultant mixture is encapsulated.

BAA prepared in accordance with Example 1 has participated in testing with 10 volunteers: pregnant women who during the examination were first diagnosed with hypothyroidism, nodules of thyroid gland and reduced immunity. For the period of pregnancy the women have been taking BAA in accordance with Example 1 thrice daily. The pregnancy of all women proceeded without any complications, and delivery went with no complications as well. Results of the analyses after 6 months of the intake of BAA in accordance with Example 1 demonstrated that this BAA has led the thyroid gland to euthyroid state and substantially improved immunity. The usage of BAA did not exert any side effects on pregnant women.

The same results have been obtained after testing the BAA with food, where the quantitative ratio of the components was chosen from within claimed range.

Thus, the disclosed BAA owing to the effectively selected qualitative and quantitative ratio of the components compounds allows providing an effective prophylactic of thyroid gland disorders and raising the immunological status without any side effects.

The BAA can be produced in a powdered, tableted or capsular form, as well as in the form of aqueous alcoholic extract and forms made on the basis of this extract, more specifically: powder, tablets or capsules.

1. (canceled)

2. A biologically active dietary additive for the normalization of the thyroid function comprises white cinquefoil, Echinacea purpurea, and Laminaria which are in the mass percent ratio of:
white cinquefoil 10-75,
Echinacea purpurea 10-50, and
Laminaria 10-80.

3. The biologically active dietary additive as claimed in claim 2, wherein roots of white cinquefoil are used.

4. The biologically active dietary additive as claimed in claim 2, wherein rhizomes of white cinquefoil are used.

5. The biologically active dietary additive as claimed in claim 2, wherein herb of white cinquefoil are used.

6. The biologically active dietary additive as claimed in claim 2, wherein a mixture of roots, rhizomes, and herb of white cinquefoil is used.
